(19) United States
(12) Patent Application Publication
(10) Pub. No.: US 2003/0130698 A1
(43) Pub. Date: Jul. 10, 2003

(54) Method for screening therapeutic agents for modulation of ejaculatory response

(76) Inventor: Klim King, Chapel Hill, NC (US)

Correspondence Address: INTELLECTUAL PROPERTY / TECHNOLOGY LAW, PO BOX 14329, RESEARCH TRIANGLE PARK, NC, 27709, US

(21) Appl. No.: 10/335,834

(22) Filed: Jan. 2, 2003

Related U.S. Application Data
(60) Provisional application No. 60/345,215, filed on Jan. 2, 2002.

(51) Int. Cl. A61N 1/18

(52) U.S. Cl. 607/3

(57) ABSTRACT

A device and method for screening therapeutic agents for modulation of sexual stimulation, including delaying ejaculatory response, damping sexual response and increasing sexual response. The method of the present invention is particularly useful for screening candidate therapeutic agents for modulating/delaying ejaculatory response, the method comprising: (a) stimulating control subjects in a physiological region that effects sexual response with an electroejaculation probe according to a predetermined stimulating protocol; (b) determining ejaculation events in the control subjects; (c) administering to testing subjects a candidate therapeutic agent for modulating sexual stimulation; (d) stimulating the testing subjects with the electroejaculation probe according to step (a); and (e) determining the ejaculation events in testing subjects and comparing the results with the ejaculation events in the control subjects to determine the efficacy of the candidate therapeutic agent.

### CROSS REFERENCE TO RELATED APPLICATION

This application claims priority from U.S. Provisional Patent Application No. 60/345,215 filed on Jan. 2, 2002.

### BACKGROUND OF THE INVENTION

1. Field of the Invention

The present invention relates to devices and methods for screening therapeutic agents, and particularly, to determining effectiveness of therapeutic agents for modulating ejaculatory response in male test subjects, including, without limitation, delaying ejaculatory response, damping sexual response or increasing sexual response.

2. Description of the Related Art

Premature ejaculation is one of the most common male sexual dysfunctions, estimated to affect up to 40% of men, irrespective of age. Premature ejaculation is defined as a persistent or recurrent ejaculation with minimal sexual stimulation before, on or shortly after penetration, which can lead to social difficulties, such as asthenia due to the loss of self-confidence, as well as domestic discord.

The reasons for premature ejaculation are generally thought to include a malfunction of the repressor center due to the fatigue of nervous transmission, hypersensitivity of a specific site due to genital disorders, hormonal disorders, physical problems and the like. It is believed that the premature ejaculation is generally caused by a complex interaction of the above mentioned reasons or by a loss of cooperation among the related sexual nerve centers.

Premature ejaculation has been conventionally treated with psychotherapy. Psychotherapy includes behavioral dual team sex therapy, according to Master & Johnson protocol, and individual psychotherapy, according to Rifelli and Moro, Sessuologia Clinica, Bologna, 1989. Psychotherapy requires sexual training for a long period of time that involves discussions and cooperation with a physician and the patient and/or his partner, and the success rate for psychotherapy is unsatisfactorily low.

Therefore, drug therapy is now more widely used as a premature ejaculation combating approach in place of psychotherapy. A variety of therapeutic drugs have been developed, including clomipramine, phenoxybenzamine, sertraline, alfuzosine, terazosine, fluoxetine, sildenafil citrate, etc. However, these drugs may not be effective for all patients, and the side effects of these drugs can halt treatment or impair patient compliance. Disease states or adverse interactions with other drugs may contraindicate the use of these compounds or require lower dosages that may not be effective to delay the onset of ejaculation. Additionally, the stigma of mental illness associated with antidepressant therapy can discourage patients from beginning or continuing such treatments.

Thus, the art continues to search for improved pharmaceutical agents for modulation of ejaculatory response. However, this search is hindered by a lack of reproducible methods for assessing the efficacy of candidate therapeutic agents. It would therefore be an advance in the art to provide a method for screening potential therapeutic agents that effect ejaculatory response.

### SUMMARY OF THE INVENTION

The present invention relates in one aspect to a method of testing and determining efficacy of therapeutic agents to modulate sexual stimulation in a male testing subject, e.g., delaying ejaculatory response, damping sexual response or increasing sexual response, the method comprising:

(a) stimulating control subjects in a physiological region that effects sexual response with an electroejaculation probe according to a predetermined stimulating protocol;

(b) determining ejaculation events in the control subjects;

(c) administering to testing subjects a candidate therapeutic agent for modulating sexual stimulation;

(d) stimulating the testing subjects with the electroejaculation probe according to the protocol of step (a); and

(e) determining the ejaculation events in testing subjects and comparing the results with the ejaculation events in the control subjects to determine the efficacy of the candidate therapeutic agent.

Another aspect of the present invention relates to determining the efficacy of a therapeutic agent to delay ejaculation in a subject, comprising the following steps:

(a) stimulating control subjects in a physiological region that effects sexual response with an electroejaculation probe according to a predetermined stimulating protocol for a predetermined number of times;

(b) determining the percentage of ejaculation events as the control ejaculation percentage;

(c) administering to test subjects a candidate therapeutic agent for delaying ejaculation;

(d) stimulating the test subjects with the electroejaculation probe while under the influence of the proposed therapeutic agent according to the protocol of step (a);

(e) determining the percentage of ejaculation events as the test ejaculation percentage;

(f) comparing the control ejaculation percentage to the test ejaculation percentage, wherein a reduction in the test ejaculation percentage relative to the control ejaculation percentage indicates an efficacious therapeutic agent for delaying ejaculation.

Another aspect of the present invention relates to a method of determining dose dependency of an ejaculation reduction effect of a therapeutic agent for combating premature ejaculation, comprising the steps of:

(a) stimulating control subjects in a physiological region that effects sexual response with an electroejaculation probe according to a predetermined stimulating protocol;

(b) determining ejaculation events in the control subjects;

(c) administering to testing subjects a candidate therapeutic agent for modulating sexual stimulation to testing subjects;

(d) stimulating the testing subjects with the electroejaculation probe according to the protocol of step (a); and

(e) determining the ejaculation events in testing subjects and comparing the results with the events in the control subjects;

(f) repeating steps (c)-(e) for multiple times, wherein at each time a different dose of the candidate therapeutic agent is administered to the subject, and an ejaculation percentage is calculated for each dose;

(g) comparing the ejaculation percentages of different doses of the therapeutic agent with the control ejaculation percentage as well as with one another, to determine dose dependency of ejaculation reduction effect of the therapeutic agent.

Yet another aspect of the present invention relates to a method for determining ejaculation-delaying effect of a premature ejaculation therapy in a subject, comprising the steps of:

(a) stimulating the subject to ejaculation with an electroejaculation probe according to a predetermined stimulating protocol;

(b) establishing a baseline stimulating duration for inducing ejaculation in the subject, according to data collected in step (a);

(c) thereafter subjecting the subject to the premature ejaculation therapy while the subject is free of stimulation with the electroejaculation probe;

(d) while the subject is under the influence of such premature ejaculation therapy, stimulating the subject to ejaculation with said electroejaculation probe according to the predetermined stimulating protocol;

(e) establishing a stimulating duration under treatment, according to data collected in step (d);

(f) comparing the stimulating duration under treatment of step (e) with the baseline stimulating duration of step (b), to determine the ejaculating-delaying effect of the premature ejaculation therapy.

Various other aspects, features and embodiments of the invention will be more fully apparent from the ensuing disclosure and appended claims.

### BRIEF DESCRIPTION OF THE DRAWINGS

FIG. 1 is a longitudinal cross-sectional view of an electric bipolar rectal probe used for inducing ejaculation, according to one embodiment of the present invention.

FIG. 2 is a graphic illustration of frequency dependency of the ejaculation response induced by stimulation via an electroejaculation probe.

FIG. 3 is a graphic illustration of dose dependency of the premature ejaculation-reducing effect of a particular therapeutic agent, SNC-80.

### DETAILED DESCRIPTION OF THE INVENTION, AND PREFERRED EMBODIMENTS THEREOF

The present invention relates to a device and method for screening therapeutic agents for modulation of sexual stimulation including, delaying ejaculatory response, damping sexual response and increasing sexual response. Preferably, the method and device provides means to measure the ejaculation delaying/inhibiting effect of a premature ejaculation therapy on male mammalian animals. Particularly, the method of the present invention is useful for screening candidate therapeutic agents for modulating/delaying ejaculatory response.

The premature ejaculation therapy, whose efficacy can be determined by the method of the present invention, can be either psychotherapy or drug therapy. Preferably, the premature ejaculation therapy is drug therapy that comprises the step of administering to the subject of a therapeutic agent for combating premature ejaculation. Such therapeutic agent may comprise any candidate drugs or chemical compounds with contemplated premature ejaculation combating effect.

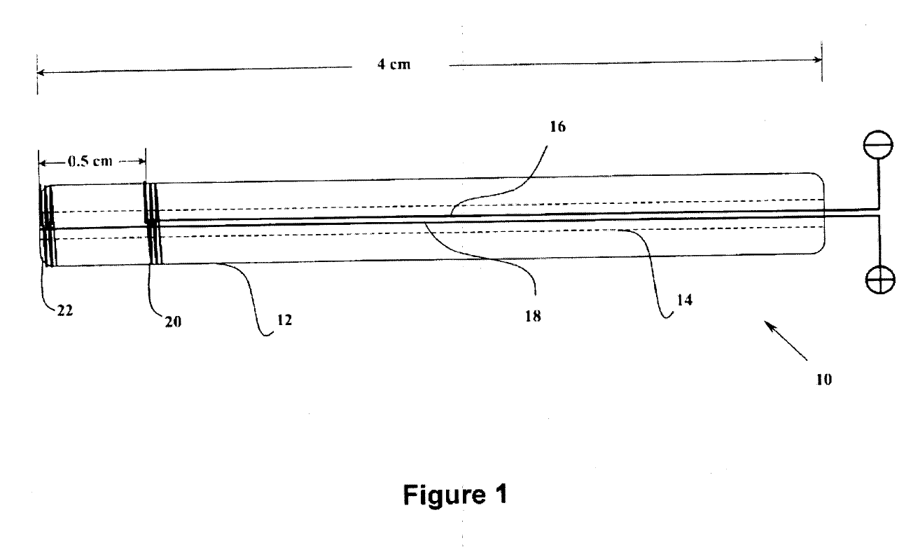

The electroejaculation probe used for practicing the present invention, preferably is an electric bipolar rectal probe 10, as shown in FIG. 1, for stimulating a male testing subject. Referring to FIG. 1, the bipolar rectal probe 10 comprises an elongated body 12 formed of any non-conductive and insulating material, such as wood, glass, natural rubber, silicone, polyurethane, polyvinyl chloride, etc. Preferably, the elongated body 12 is formed of natural rubber for bendability and ease of insertion. Preferably, the bipolar rectal probe is a tubular device, comprising a first end for insertion into rectum of the testing animal and a second end connected to a power source. The probe is of sufficient dimensions for insertion into the rectum of the testing animal. More preferably, when the tubular device is used with mice, the elongated body is approximately 3 to 5 cm in length and an outer diameter of about 0.2 to about 0.4 cm and an inner diameter of about 0.05 to about 0.08 cm (the inner diameter line is indicated by reference number 14). Analogous probes of appropriate size may be fabricated and employed in the general practive of the invention for various other subjects, e.g., mammalian subjects including human as well as non-human mammals.

The elongated tubular body 12 further comprises a cathode 16 and anode 18, which are formed of a conductive material and positioned in a center lumen that is aligned with the with the longitudinal axis of the device. Any conductive material may be used to provide electrical current to the point of contact of the electrodes, including, but not limited to, platinum, copper, conductive polymers and the like.

Anode 18 extends from the second end to the first end through the center lumen and extending to and through the outer surface of the first end to provide an exterior anode terminal 22. Preferably, the exterior anode provides a contact area around the circumference of the first end. Cathode 16 extends to at least a middle portion of the elongated body 12, but does not extend to the first end. The cathode extends through the wall surface of the tubular body thereby forming an exterior cathode terminal 20 at such middle portion placement. Preferably, the cathode provides a contact area around the circumference of the middle portion of the tubular body. The cathode terminal 20 is positioned away from the anode terminal 22, at a sufficient distance to position a sensitive stimulating region of the testing subject between and/or proximate to the electrodes, thereby allowing stimulating current to travel between the electrodes and through the sensitive stimulating region. The exact distance between the exterior electrodes will be dependent on the size of the testing subject and can be determined by one skilled in the art.

The cathode 16 and anode 18 are connected at the second end to electrical and electronic components including a signal generator with associated control circuitry, and a power supply and if desired associated with a printed circuit board for programmable frequencies and voltage regimes. The electrical and electronic components may include, an off-on switch to activate the probe 10. As an alternative, the probe may be activated by means of a radio frequency or other remote signal.

The bipolar rectal probe as shown in FIG. 1 is used for practicing the method of the present invention. An ordinary person skilled in the art may readily modify the size of such bipolar rectal probe of FIG. 1 to make it suitable for stimulation of a particular test subject.

In operation, the probe 10 is lubricated and inserted into the rectum of the restrained animal. Conductive material of the band-like exterior electrodes 16 and 18 contact the body of the subject thereby allowing electrical current to passed between the electrodes 16 and 18 thereby stimulating nerves and muscles involved in erection and ejaculation. Advantageously, the placement of the electrodes at the first end and approximately a middle section of the probe provides for placement of the sensitive stimulating region of the test subject proximate to the electrodes.

Identification of the physiological regions to be stimulated by the probe 10 is accomplished by applying electrical energy which simulates the erection-stimulating signal ultimately to be produced by the probe 10. Such applications, when made at sensitive physiological regions, cause palpable or visible physical reactions which are signs of incipient erection. It has been found in laboratory animals that the application of electrical stimuli to critical spots of the prostate yields the desired result, while other regions show relative insensitivity to the application of the stimulus. One of the advantages of the present probe and method flow from the ability of the probe 10 to apply energy directly to only those specific regions which have been ascertained to provide the desired result. When desirable spots have been identified and noted, the desired locations of electrodes may be ascertained by measurement, so that when the probe 10 is positioned for use, the electrodes are proximate to the efficacious spots on testing animal.

To conduct the testing method of the present invention, the equipment defined herein is utilized. An electrical power supply stimulator is adapted by electronic circuitry according to electrical setups well known to one skilled in the art to deliver a current in a 20 to 60 Hz periodic waveform. More preferably, the current is delivered in a range from about 40 to about 45 Hz. The circuit, which has interconnection with the probe, is designed to generate stimulation in the amount from 1 to about 10 volts, and more preferably from about 3 to 7 volts.

Careful notation is made of the stimulation protocol that includes a gradual increase in voltage at either a constant frequency or a variable frequency. Preferably, the voltage is increased in small increments after a stimulating pulsing session. For example, at a specific voltage, there is administered four pulses of electrical stimulation lasting a predetermined length with a predetermined non-stimulating interval. This gradual increase of voltage during each pulse-stimulation period is important to induce ejaculation in a mouse, since this inventor found that stimulation of a testing subject at a fixed voltage, even with 20-30 pulse stimulations did not result in ejaculation.

The predetermined stimulation protocol used in the practice of the present invention comprises a set pattern of electrostimulations conducted at specific effective frequencies and voltages. The design of such stimulation protocol helps to generate reproducible ejaculation data that are important for accurately assessing the efficacy of candidate therapeutic agents in combating premature ejaculation.

Particularly, the stimulation protocol of the present invention comprises multiple operating/stimulating cycles of alternating stimulation periods and rest periods, while operating voltage of each operating cycle increases in comparison with that of the immediate preceding operating cycles. The starting operating voltage is generally set within the range of from about 1 volts to about 5 volts with increasing increments to a terminal voltage of at least one increment greater than the starting voltage, and preferably the terminal voltage is from about 2 to about 10 volts. More preferably, the testing regime includes a starting voltage of about 3 volts with an increase in voltage of at least one increment greater that the starting voltage until ejaculation or a terminal voltage is reached. Preferably the terminal voltage is about 7 to 8 volts. The increment of increased voltage should be in a sufficient amount to make at least a noticeable difference in the ejaculation effect and preferably from about 0.1 to 1 volt. Understanding that one skilled in the art can determine the ideal voltage range depending on the size of the testing animal.

As shown below in Table 1, a predetermined stimulating protocol is conducted according to the following voltage program wherein the testing regime was conducted between a starting voltage of 3 volts, a terminal voltage of 8 volts with increased increments of 0.5 volts. It should be noted that this test is one of a multiplicity of tests that include the starting voltage of 3 volts with different terminal voltages, such as 5 volt, 7 volt or 8 volts due to ejaculation events. To have a complete picture of ejaculation events at different terminal voltages, each test will end when ejaculation occurs at a specific voltage, if the ejaculation event is evident due to antegrade ejaculation. In the alternative, if retrograde ejaculation is more likely the norm, a terminal voltage is selected and to determine ejaculation the test animal is sacrificed.

TABLE 1

| Operating | | Duration | Voltage |
|---|---|---|---|
| Cycle | Period | (seconds) | (volts) |
| Operating Cycle 1 | 4 Stimulations | 2 | 3 |
| | 4 Rest Period between | 2 | 3 |
| | each stimulation event | | |
| Operating Cycle 2 | 4 Stimulations | 2 | 3.5 |
| | 4 Rest Periods between | 2 | 3.5 |
| | each simulation event | | |
| Operating Cycle 3 | 4 Stimulations | 2 | 4 |
| | 4 Rest Periods between | 2 | 4 |
| | each stimulation event | | |
| Operating Cycle 4 | 4 Stimulation | 2 | 4.5 |
| | 4 Rest Periods between | 2 | 4.5 |
| | each stimulation event | | |
| Operating Cycle 5 | 4 Stimulations | 2 | 5 |
| | 4 Rest Periods between | 2 | 5 |
| | each stimulation event | | |
| Operating Cycle 6 | 4 Stimulations | 2 | 5.5 |
| | 4 Rest Periods between | 2 | 5.5 |
| | each stimulation event | | |
| Operating Cycle 7 | 4 Stimulations | 2 | 6 |
| | 4 Rest Periods between | 2 | 6 |
| | each stimulation event | | |
| Operating Cycle 8 | 4 Stimulations | 2 | 6.5 |
| | 4 Rest Periods between | 2 | 6.5 |
| | each stimulation period | | |
| Operating Cycle 9 | 4 Stimulations | 2 | 7 |
| | 4 Rest Periods between | 2 | 7 |
| | each stimulation period | | |
| Operating Cycle 10 | 4 Stimulations | 2 | 7.5 |
| | 4 Rest Periods between | 2 | 7.5 |
| | each stimulation period | | |
| Operating Cycle 11 | 4 Stimulations | 2 | 8 |
| | 4 Rest Periods between | 2 | 8 |
| | each stimulation period | | |

It is also discovered that the occurrence of ejaculation in male mouse is highly dependent on the frequency used to stimulating the mouse. For instance, the voltage program as described herein above is especially effective in inducing ejaculation in male mice, with an effective rate of 100% when operated under a oscillating current of 60 Hz or higher.

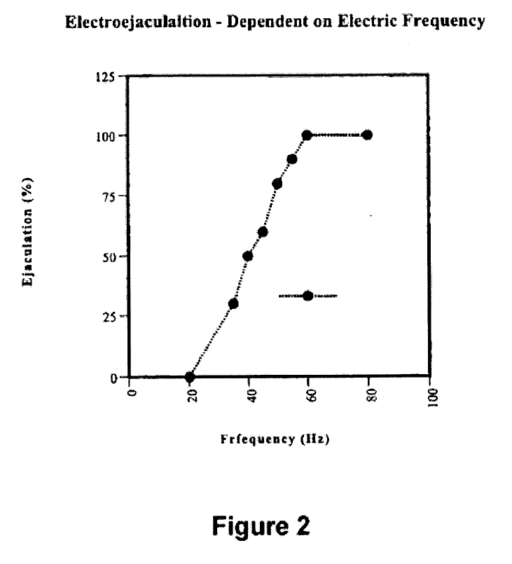

When stimulating 10 mice through the voltage program as shown in Table 1 at an operating frequency of 20 Hz or lower, none of the mice ejaculate. When the operating frequency increased from 30 to 70 Hz, the occurrence of successful ejaculation also increased, which is indicated by emission of a white coagulum from the penis of the male mouse under stimulation. When the operating frequency of the electric stimulation is between 30 to 45 Hz, the ejaculation rate showed a linear dependency on the frequency, as shown in FIG. 2.

It is therefore preferable to practice the method of the present invention at an operating frequency within the range from about 20 Hz to about 80 Hz. More preferably, the operating frequency is within a range selected from about 35 to about 50 Hz, and most preferably from about 40 to about 45 Hz.

To show that the present system and electrical stimulation regime is effective to assay the effects of a candidate therapeutic agent on ejaculation, certain delta opioid receptor agonist compounds were tested to determined if the compounds had premature ejaculation inhibiting/reducing effect on male mice.

Delta opioid receptors are present in the central and peripheral nervous systems of many species including man. The delta opioid receptor has been identified as having a role in many bodily functions, such as circulatory and pain systems, immunomodulatory activities, and gastrointestinal disorders. Agonists are agents that recognize and bind to the delta receptors thereby affecting biochemical and/or physiological pathways. One of the major neuronal effects of opioid receptor activation is blocking the release and liberation of neurotransmitters. The neurotransmitter adrenaline is liberated by postganglic sympathetic nerve and which initiates the contraction of smooth muscle surrounding seminal vesicle and prostate gland that leads to semen emission. Likewise, the parasympathetic nerve liberates a neurotransmitter that initiates contraction of the bulbocarvernous muscle surround the penis which leads to forcible ejection of semen from the urethra. While not wishing to be bound by any specific mechanism of action, it is believed that the activation of the delta opioid receptor leads to an inhibiting release of adrenaline or acetylcholine from sympathetic and parasympathetic nerve endings, and consequently prevents smooth muscle from contraction with a concomitant delay of ejaculation. It is noteworthy to point out that heretofore no reference appears in the literature about any possible use of delta opioid receptor agonists, either peptidic or non-peptidic, in treatment of premature ejaculation. Thus, the use of delta opioid receptor agonist compounds were considered the perfect candidate for implementing the probe and stimulating testing methods of the present invention.

The delta opioid receptor agonists screened by the device and methods of the present invention include both peptidic and non-peptidic compounds. The delta opioid receptor agonist used for testing of the present invention may include non-peptidic diarylmethylpiperazine compounds and peptidic compounds, such as Deltorphin I, [D-Ala²]-Deltorphin II, biphalin, DADLE, DPDPE, and DSLET.

[D-Ala²]-Deltorphin II (Tyr-D-Ala-Phe-Glu-Val-Val-Gly-NH₂) is a selective peptide agonist for delta opioid receptors.

Biphalin is an opioid agonist that stimulates both mu and delta opioid receptors in the central nervous system.

Deltorphin I (Tyr-D-Ala-Phe-Asp-Val-Val-Gly-NH₂) is a peptide delta opioid receptor agonist. DADLE which is also named [D-Ala², D-Leu⁵] enkephalin is a modified delta opioid receptor agonist.

DPDPE, which is also called D-Pen₂, D-Pen₅-enkephalin, is a selective delta opioid receptor agonist formed by chemically modifying an endogenous opioid.

DSLET, which is also called [D-Ser², Leu⁵]enkephalyl-Thr, is another peptide delta opioid receptor agonist.

To investigate the efficacy of delta opioid receptor agonists as a treatment for premature ejaculation, intact conscious male mice were electrically stimulated subsequent to the administration of a delta opioid receptor agonist to determine if a delay in ejaculation was effected when compared to administration of a placebo

General Materials and Methods

Male, CD-1 mice (20-30 g) were housed in groups of ten (10) in Plexiglas® chambers with food and water available before any procedure. Animals were maintained on a 12 hour light/dark cycle in a temperature-controlled animal colony. Studies were carried out in accordance with the Guide for the Care and Use of Laboratory animals as adopted and promulgated by the National Institutes of Health.

The electric bipolar rectal probe, as shown in FIG. 1, was used for stimulating the subjects. Specifically, the bipolar electrode probe is an approximately 5 cm long tube having an outer diameter of approximately 0.2388 cm with an inner diameter of approximately 0.0787 cm and a wall thickness of 0.1994 cm.

The first testing compound was a delta opioid receptor agonist (SNC-80) was purchased for Tocris Cookson, Inc., Ellisville, Mo., U.S.A. SNC-80, having an IUPAC name of (+)-4-[(αR)-α-((2S,5R)-4-Allyl-2,5-dimethyl-1-piperazinyl)-3-methoxybenzyl]-N,N-diethyl benzamide, is a highly selective and potent non-peptide delta opioid receptor agonist, having a 2000-fold selectivity over mu opioid receptors. SNC-80 was dissolved in 5% dextrose injection solution. Equal molar of HCL was added when the manufacturer packaged the compound in a base form. The placebo was prepared in a similar manner without the test drug 10 minutes after the injection, the test mouse was subjected to electrostimulation. Ten mice were tested for each dose level, the order of each mouse receiving different doses was blinded and in a random manner.

A testing mouse was restrained in a cone bag with rear legs extending from the bag. Excess fecal matter was removed from the rectum and a lubricated bipolar electrode was inserted approximately 2.5 into the rectum. An oscillating current of 40 Hz was utilized for electrical stimulation starting at 3 volts with a gradual increase to 8 volts by increments of 0.5 volts, as shown in Table 1. The stimulation regime included 4 stimulating events for each voltage lasting 2 seconds with a rest period of 2 seconds between stimulating events. A white coagulum ejaculate from the penis indicated a successful ejaculation. If ejaculation occurred before reaching the 8 volt terminal voltage, the testing was stopped.

Testing was conducted with injection of SNC-80 or a buffer vehicle for the control group. SNC-80 is a highly selective delta receptor agonist which has been found to block the contraction of mouse vas deferens smooth muscle in vitro which is one of the tissue components in the ejaculation system. Each mouse was subjected to electroejaculation after subcutaneous administration of SNC-80 at different doses of 0, 0.1, 0.2, 0.3, 0.5 and 1 mg/kg. A minimum of 10 mice were used at each dose level. Each does response is the average of two to three independent experiments ±SEM. For electroejaculation test, dose-response lines were constructed as an accumulated ejaculation at a specified voltage. Student's t-test was used to assess unpaired comparison, with p<0.06 indicating significance.

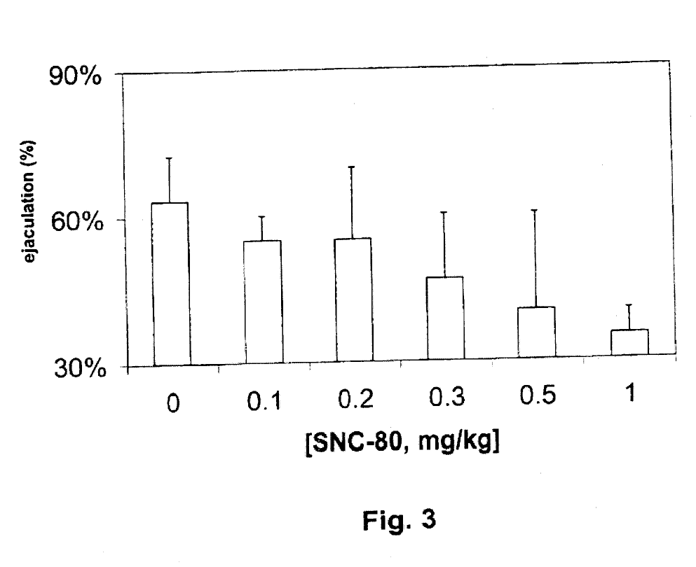

As shown in FIG. 3, when the frequency of the electric stimulation was set at 40 Hz and a terminal voltage of 8 volts, a dose of 1 mg/kg of SNC-80 significantly reduced the occurrence of ejaculation in tested mice as compared to that of the control group. This reduction of ejaculation by SNC-80 is specific, as the compound works in a dose dependent manner and the inhibitory effect diminished as the concentration reduced to 0.1 mg/kg.

Further testing was conducted on a multiple diarylmethylpiperazine compounds according to the testing procedure setforth above wherein the stimulating protocol comprised multiple operating cycles of alternating stimulation periods and rest periods during which the operating voltage of each operating cycle increased in comparison with that of the immediately preceding operating cycle. Electroejaculation stimulation was conducted at 35 Hz oscillating frequency, starting at 3 volts with a gradual increase to 8 volts by increments of 0.5 volts and occurred about 20 minutes after oral administration. The results, showing ejaculation inhibition relative to that of the control group, are compiled in Table 2.

TABLE 2

| | Effective | |
|---|---|---|
| | Dose | Ejaculation |
| Compound | (mg/kg) | Inhibition % |
| 3-((alpha-R)-alpha-((2S,5R)-4-Allyl-2,5-dimethyl-1-piperazinyl)-4- | 0.01 | 45% |
| (diethylamino-carbonyl)benzyl)phenoxyacetic acid | | |
| 3-((alpha-R)-alpha-((2S,5R)-4-Benzyl-2,5-dimethyl-1-piperazinyl)-4- | 3 | 66% |
| (diethylaminocarbonyl)benzyl)phenoxyacetic acid | | |
| 4-((alpha-R)-alpha-((2S,5R)-2,5-Dimethyl-4-(4-fluorobenzyl)-1- | 3 | 38% |
| piperazinyl)-3-methoxybenzyl)-N,N-diethylbenzamide | | |
| 4-(alpha-R)-alpha-((2S,5R)-4-(Cyclopropylmethyl)-2,5-dimethyl- | 0.3 | 60% |
| 1-piperazinyl)-3-hydroxybenzyl)-N,N-diethylbenzamide | | |
| 4-((alpha-R)-alpha-((2S,5R)-2,5-Dimethyl-4-(3-fluorobenzyl)-1- | 3 | 60% |
| piperazinyl)-3-hydroxybenzyl)-N,N-diethylbenzamide | | |
| 4-((alpha-S)-alpha-((2S,5R)-2,5-Dimethyl-4-(4-hydroxybenzyl)-1- | 3 | 50% |
| piperazinyl)-benzyl)-N,N-diethylbenzamide | | |
| 4-(alpha-R)-alpha-((2S,5R)-4-Benzyl-2,5-dimethyl-1 piperazinyl)-3- | 3 | 33% |
| methoxybenzyl)-N,N-diethylbenzamide | | |
| 4-((alpha-R)-alpha-((2S,5R)-2,5-Dimethyl-4-(2-fluorobenzyl)-1- | 10 | 50% |
| piperazinyl)-3-methoxybenzyl)-N,N-diethylbenzamide | | |
| 4-[(R)-((2R,5S)-4-Allyl-2,5-diimethylpiperazin-1-yl) | 0.3 | 50% |
| (3-hydroxyphenyl)methyl]-N,N-dimethylbenzenesulfonamide | | |
| 4-((alpha-R)-alpha-((2R,5 S)-2,5-Dimethyl-4-(3-fluorobenzyl)-1- | 0.4 | 75% |
| piperazinyl)benzyl)-N,N-diethylbenzamide | | |

While the invention has been described herein in reference to specific aspects, features and illustrative embodiments of the invention, it will be appreciated that the utility of the invention is not thus limited, but rather extends to and encompasses numerous other aspects, features and embodiments. Accordingly, the claims hereafter set forth are intended to be correspondingly broadly construed, as including all such aspects, features and embodiments, within their spirit and scope.

What is claimed is:

1. A method of testing and determining efficacy of a therapeutic agent to modulate sexual stimulation in a testing subject, the method comprising:
(a) stimulating control subjects in a physiological region that effects sexual response with an electroejaculation probe according to a predetermined stimulating protocol;
(b) determining ejaculation events in the control subjects;
(c) administering to testing subjects a candidate therapeutic agent for modulating sexual stimulation;
(d) stimulating the testing subjects with the electroejaculation probe according to the protocol of step (a); and
(e) determining the ejaculation events in testing subjects and comparing the results with the ejaculation events in the control subjects to determine the efficacy of the candidate therapeutic agent.

2. The method of claim 1, wherein the therapeutic agent to modulate sexual stimulation is for combating premature ejaculation.

3. The method of claim 2, wherein the therapeutic agent comprises a delta opioid receptor agonist.

4. The method of claim 3, wherein the delta opioid receptor agonist comprises a peptide compound.

5. The method of claim 3, wherein the delta opioid receptor agonist comprises a non-peptide compound.

6. The method of claim 3, wherein the therapeutic agent comprises a compound selected from the group consisting of: diarylmethylpiperazines, Deltorphin I, [D-Ala²]-Deltorphin II, DADLE, biphalin, DPDPE, and DSLET.

7. The method of claim 1, wherein the electroejaculation probe comprises an electric bipolar rectal probe.

8. The method of claim 7, wherein the electric bipolar rectal probe comprises an elongated body having a center lumen and formed of a non-conducting material, wherein the elongated body has a first and second end, an anode exteriorly located on the first end of the elongated body, and a cathode exteriorly located in spaced relationship to the anode at a sufficient distance to position the physiological region proximate to the electrodes for passage of stimulating current through the physiological region.

9. The method of claim 8, wherein both the anode and cathode comprise platinum as a material of construction.

10. The method of claim 1, wherein the predetermined stimulating protocol is conducted at an operating frequency within a range of from about 35 Hz to about 45 Hz.

11. The method of claim 2, wherein a reduction in the test ejaculation percentage relative to the control ejaculation percentage indicates an efficacious therapeutic agent for delaying ejaculation.

12. The method of claim 1, wherein the predetermined stimulating protocol comprises multiple operating cycles of alternating stimulation periods and rest periods, and wherein operating voltage of each operating cycle increases in comparison with that of an immediately preceding operating cycle.

13. The method of claim 12, wherein the operating voltage is within a range of from about 3 volts to about 8 volts.

14. The method of claim 12, wherein the predetermined stimulating protocol is conducted at an operating frequency of about 40 Hz, and comprising the steps of:
(a) conducting an initial operating cycle at an operating voltage of about 3 volts, wherein said first operating cycle comprises four stimulation periods and four rest periods, and wherein duration of each stimulation/rest period is about two seconds; and
(b) conducting additional operating cycles, as specified in step (a), except at a different operating voltage, wherein the operating voltage of each additional operating cycle increases about 0.5 volt in comparison with that of an immediately preceding operating cycle, until ejaculation occurs or the operating voltage reaches about 8 volts.

15. A method of determining dose dependency of an ejaculation reduction effect of a therapeutic agent for combating premature ejaculation, comprising the steps of:
(a) stimulating control subjects in a physiological region that effects sexual response with an electroejaculation probe according to a predetermined stimulating protocol;
(b) determining ejaculation events in the control subjects;
(c) administering to testing subjects a candidate therapeutic agent for modulating sexual stimulation;
(d) stimulating the testing subjects with the electroejaculation probe according to the protocol of step (a); and
(e) determining the ejaculation events in testing subjects and comparing the results with the ejaculation events in the control subjects;
(f) repeating steps (c)-(e) for multiple times, wherein at each time a different dose of the candidate therapeutic agent is administered to the subject, and an ejaculation percentage is calculated for each dose;
(g) comparing the ejaculation percentages of different doses of the therapeutic agent with the control ejaculation percentage as well as with one another, to determine dose dependency of ejaculation reduction effect of the therapeutic agent.

16. The method of claim 15, wherein the therapeutic agent comprises a delta opioid receptor agonist.

17. The method of claim 16, wherein the delta opioid receptor agonist comprises a peptide compound.

18. The method of claim 16, wherein the delta opioid receptor agonist comprises a non-peptide compound.

19. The method of claim 16, wherein the therapeutic agent comprises a compound selected from the group consisting of: SNC-80, SNC-121, diarylmethylpiperazines, Deltorphin I, [D-Ala²]-Deltorphin II, biphalin, DPDPE, and DSLET.

20. The method of claim 15, wherein the electroejaculation probe comprises an electric bipolar rectal probe.

21. The method of claim 20, wherein the electric bipolar rectal probe comprises an elongated body having a center lumen and formed of a non-conducting material, wherein the elongated body has a first and second end, an anode exteriorly located on the first end of the elongated body, and a cathode exteriorly located in spaced relationship to the anode at a sufficient distance to position the physiological region proximate to the electrodes for passage of stimulating current through the physiological region.

22. The method of claim 15, wherein the predetermined stimulating protocol is conducted at an operating frequency within a range of from about 30 Hz to about 70 Hz.

23. The method of claim 15, wherein the predetermined stimulating protocol is conducted at an operating frequency within a range of from about 40 Hz to about 45 Hz.

24. The method of claim 15, wherein the predetermined stimulating protocol comprises multiple operating cycles of alternating stimulation periods and rest periods, and wherein operating voltage of each operating cycle increases in comparison with that of an immediately preceding operating cycle.

25. The method of claim 24, wherein the operating voltage is within a range of from about 3 volts to about 8 volts.

26. The method of claim 25, wherein the predetermined stimulating protocol is conducted at an operating frequency of about 40 Hz, and comprising the steps of:
(a) conducting an initial operating cycle at an operating voltage of about 3 volts, wherein said first operating cycle comprises four stimulation periods and four rest periods, and wherein duration of each stimulation/rest period is about two seconds; and
(b) conducting additional operating cycles, as specified in step (a), except at a different operating voltage, wherein the operating voltage of each additional operating cycle increases about 0.5 volt in comparison with that of an immediately preceding operating cycle, until the operating voltage reaches about 8 volts.

27. A method for determining ejaculation-delaying effect of a premature ejaculation therapy in a subject, comprising the steps of:
(a) stimulating the subject to ejaculation with an electroejaculation probe according to a predetermined stimulating protocol;
(b) establishing a baseline stimulating duration for inducing ejaculation in said subject, according to data collected in step (a);
(c) thereafter subjecting the subject to said premature ejaculation therapy while the subject is free of stimulation with said electroejaculation probe;
(d) while the subject is under the influence of said premature ejaculation therapy, stimulating the subject to ejaculation with said electroejaculation probe according to said predetermined stimulating protocol;
(e) establishing a stimulating duration under treatment, according to data collected in step (d); comparing the stimulating duration under treatment of step (e) with the baseline stimulating duration of step (b), to determine the ejaculating-delaying effect of the premature ejaculation therapy.

28. A bipolar rectal probe for sexual stimulation of a male testing subject comprising:
an elongated body having a center lumen aligned with the longitudinal axis of the elongated body, the elongated body formed of a non-conductive material of sufficient dimensions for rectal insertion into the testing subject, the elongated body having a first and second end, the first end for rectal insertion into the testing subject and a second end proximate to a power source;
an anode formed of a conductive material connected to the power source and extending from the second end to the first end of the elongated body through the center lumen and extending to and through an outer surface of the first end to provide an exterior anode around the circumference of the first end;
a cathode formed of a conductive material connected to the power source and extending from the second end to a middle section of the elongated body through the center lumen and extending to and through an outer surface of the middle section of the elongated body to provide an exterior cathode anode around the circumference of the middle section, wherein the cathode is spaced from the anode at a sufficient distance to locate a sensitive stimulating region of the testing subject between the electrodes, thereby allowing stimulating current to travel between the electrodes and through the sensitive stimulating region.
